(19) United States
(12) Patent Application Publication
(10) Pub. No.: US 2023/0242961 A1
(43) Pub. Date: Aug. 3, 2023

(54) Production of Soluble Recombinant Protein

(71) Applicant: Fina BioSolutions, LLC, Rockville, MD (US)

(72) Inventors: Min-Ju Chang, Gaithersburg, MD (US); Natalia Oganesyan, North Potomac, MD (US); Andrew Lees, Silver Spring, MD (US)

(73) Assignee: Fina BioSolutions, LLC, Rockville, MD (US)

(21) Appl. No.: 17/797,851

(22) PCT Filed: Mar. 12, 2021
(86) PCT No.: PCT/US2021/022126

§ 371 Date: Aug. 5, 2022

Related U.S. Application Data
(60) Provisional application No. 63/152,954, filed on Feb. 24, 2021.
(60) Provisional application No. 62/900,083, filed on Sep. 13, 2019.

(51) Int. Cl. C12P 21/02; C12N 9/48; C12N 15/70

(52) U.S. Cl. CPC C12P 21/02; C12N 9/485; C12N 15/70; C12Y 304/11018; C12N 2800/101

(57) ABSTRACT

The invention is directed to methods and compositions for the expression and purification of products such as peptides and proteins in microorganisms. In particular, pre-products are expressed recombinantly, wherein the cytoplasm of the microorganism alters the expressed pre-products to produce products in an active/final or otherwise desirable form. Alterations associated with expression of a desired recombinant product include shifting of the redox state of the cytoplasm to allow proper protein folding, site-directed cleavage of pre-proteins to activate the protein, site-directed cleavage of an unwanted methionine from the N terminus of the protein, and/or one or more ligations to form desired protein configurations, all within the same cell.

### RIGHTS IN THE INVENTION

The invention was made with United States Government support under Grant No. 1R43AI148018-01A1 FAIN: R43AI148018, awarded by the National Institutes of Health, and the U.S. Government has certain rights in the invention.

### REFERENCE TO RELATED APPLICATIONS

This application claims priority to U.S. Provisional Application No. 63/152, 954 filed Feb. 24, 2021; U.S. Provisional Application No. 62/990,083 filed Mar. 16, 2020; and U.S. Application No. 16/819,775 filed Mar. 16, 2020, and presently pending, the entirety of each of which is specifically incorporated by reference.

### FIELD OF THE INVENTION

The invention is directed to methods and compositions to express and purify products such as peptides and proteins in microorganisms. In particular, pre-products are expressed recombinantly, wherein the cytoplasm of the microorganism alters the expressed pre-products to produce products in a final or usable form. Alterations include shifting of the redox state of the cytoplasm and site-directed cleavage and/or ligation.

### DESCRIPTION OF THE BACKGROUND

E. coli is a widely used host to produce recombinant proteins for research and therapeutic purposes. Recombinant proteins can be expressed in E. coli cytoplasm or periplasm. One limitation to cytoplasmic recombinant protein expression in E. coli is that, to initiate expression of recombinant protein in E. coli, the coding sequence of the protein should start with the ATG codon, which is translated to formyl-methionine and then processed by formylmethionine deformylase to become N-terminal methionine. Therefore, for recombinant protein expression in E. coli, the ATG codon is added to the native or mature protein sequence. During intracellular expression of recombinant protein, the N-terminal Methionine is usually excised by endogenous E. coli methionine aminopeptidase (MAP). This process is not necessarily efficient for recombinant proteins, even if the residue adjacent is optimal for cleavage, likely due to overexpression of the recombinant protein and the limited amount of MAP present. As a result, a substantial amount of the recombinant protein may have Methionine as the first amino acid. This is undesirable for most proteins as the N-terminal Methionine is not a part of the mature protein sequence. The presence of the N-terminal Methionine may also cause structural changes to a protein that affects its function. Existing methods to ensure effective cleavage of the N terminal-methionine include in vitro treatment with recombinant MAP. Another approach is adding the MAP coding sequence to the expression vector and thus co-expressing MAP along with the recombinant protein. In the latter case, co-expression of MAP may reduce expression of the desired recombinant protein. Both approaches are time-consuming and costly to implement. Thus, it would be desirable to have an E. coli expression strain where MAP was highly expressed without significantly inhibiting the recombinant protein expression. This would allow the production of increased amounts of the target protein with its native sequence in the cytoplasm.

Recombinant proteins expressed in the cytoplasm of E. coli may form insoluble inclusion bodies. Proteins in inclusion bodies may be refolded in-vitro to form soluble proteins. These proteins will contain an N-terminal methionine, which is undesirable.

The E. coli cytoplasm has a reducing environment, and recombinant proteins containing disulfide bonds are usually insoluble when expressed intracellularly. In contrast, the periplasm of E. coli has an oxidative environment. Therefore, many recombinant proteins containing disulfide bonds are secreted into the periplasm in order to ensure proper folding and solubility. The signal peptide that directs recombinant protein into periplasm is clipped off during the secretion process, resulting in the production of protein with the native amino acid sequence. However, the translocation mechanisms that direct proteins to the periplasm have limited capacity, and so periplasmic expression level of recombinant proteins is usually low. On the other hand, expression in the E. coli cytoplasm can lead to grams of recombinant proteins per liter of cell culture. Therefore, it would be desirable to be able to express soluble, properly folded disulfide-bonded proteins in the cytoplasm. Furthermore, it would be desirable if these proteins could be produced without the N-terminal Methionine.

Commercially available E. coli strains such as Origami® (EMDMillipore), Shuffle® (New England Bio) with gor-/trx- mutations, can produce soluble, intracellular proteins containing disulfide bonds, but these cell strains are crippled and do not grow to a high-density, limiting production yield. Thus, while these strains are suitable for generating research material, their low growth levels make them difficult to use commercially. Thus, a need exists for strains that express high levels of properly folded intracellular disulfide-bonded proteins that do not contain an N-terminal methionine.

### SUMMARY OF THE INVENTION

The present invention overcomes the problems and disadvantages associated with current strategies and designs and provides new compositions and methods for producing recombinant peptides and proteins.

One embodiment of the invention is directed to methods of producing recombinant peptides and proteins in bacteria comprising: expressing the protein in a bacteria containing an expression vector that encodes the protein sequence including a promoter and the bacteria also contains a gene under the control of a promoter, which is integrated into the genome of the host, wherein the polypeptide expressed by that gene facilitates the expression, folding or solubility of the recombinant protein in the cytoplasm and isolating the protein.

Another embodiment of the invention is directed to methods of producing recombinant peptides and proteins in bacteria comprising: expressing the protein in a bacteria containing an expression vector that encodes the protein sequence including a promoter and the bacteria also expresses a peptidase gene, which is integrated into the genome of the host cell, where the peptidase expression is under the control of a promoter, such that the peptidase acts on the protein expressed and removes a formyl-methionine group from the N-terminal portion of the protein; and isolating the protein. Peptidases that remove an N-terminal methionine can be referred to as Methionine amino peptidases (MAP). Preferably the integrated gene contains a ribosome binding site, an initiation codon, and an expression enhancer and/or repressor region. Preferably the recombinant cell has reduced activity of only one disulfide reductase enzyme or a reduced activity of only two disulfide reductase enzymes. Preferably the recombinant cell is an E. coli cell or a derivative or strain of E. coli, and preferably the recombinant protein expressed comprises tetanus toxin, tetanus toxin heavy chain proteins, diphtheria toxoid, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, cytokines, single chain antibodies, camelids, nanobodies and fragments, derivatives, and modifications thereof. Also preferably, the recombinant protein may be a pre-protein prorelaxin, insulin and members of the insulin-like family. Preferably the integrated gene and/or expression vector contains an inducible promoter for the peptidase. Expressing comprises inducing the inducible promoter with a first inducing agent and contains an expression vector that encodes the recombinant peptide or protein which may be inducible with a second inducing agent. Preferably the first and second inducing agents are the same, although they may be different. Preferably the first integrated gene or expression vector contains an inducible second promoter and expressing the peptidase comprises inducing the inducible second promoter with the first inducing agent. Preferably isolating comprises chromatography wherein the chromatography comprises a sulfate resin, a gel resin, an active sulfated resin, a phosphate resin, a heparin resin, or a heparin-like resin. Preferably the isolated protein expressed is conjugated with polyethylene glycol and/or a derivative of polyethylene glycol or with a polymer such as, for example, a polysaccharide, a peptide, an antibody or portion of an antibody, a lipid, a fatty acid, a small molecule, hapten or a combination thereof.

Another embodiment of the invention is directed to methods of producing a soluble or insoluble peptide comprising: expressing the peptide with a formyl-methionine group at an N-terminus of the peptide from a recombinant cell containing an expression vector that encodes the peptide and expressing a peptidase from an integrated gene of a recombinant cell that acts on the peptide expressed and removes the formyl-methionine group from the N-terminus of the peptide; and isolating the peptide.

Another embodiment of the invention is directed to methods of producing a peptide comprising: expressing the peptide with a formyl-methionine group at an N-terminus of the peptide from a recombinant cell containing an expression vector that encodes the peptide, wherein the recombinant cell has a reduced activity of one or more disulfide reductase enzymes and the expression vector contains a promoter functionally linked to a coding region of the peptide, wherein the reduced activity of one or more disulfide reductase enzymes results in a shift the redox status of the cytoplasm to a more oxidative state as compared to a recombinant cell that does not have reduced activity of one or more disulfide reductase enzymes, and expressing a peptidase from an integrated gene of a recombinant cell that acts on the peptide expressed and removes the formyl-methionine group from the N-terminus of the peptide; and isolating the peptide. Preferably the expression vector contains a ribosome binding site, an initiation codon, and an expression enhancer/repressor region. Preferably the recombinant cell has a reduced activity of only one disulfide reductase enzyme or only two disulfide reductase enzymes. Preferably the one or more disulfide reductase enzymes comprise one or more of an oxidoreductase, a dihydrofolate reductase, a thioredoxin reductase, or a glutathione reductase. Preferably the recombinant cell is an E. coli cell or a derivative or strain of E. coli and the peptide or protein comprises tetanus toxin, tetanus toxin heavy chain proteins, diphtheria toxoid, tetanus toxoid, Pseudomonas exoprotein A, Pseudomonas aeruginosa toxoid, Bordetella pertussis toxoid, Clostridium perfringens toxoid, Botulism toxin, Escherichia coli heat-labile toxin B subunit, Neisseria meningitidis outer membrane complex, Hemophilus influenzae protein D, Flagellin Fli C, cytokines, single chain antibodies, camelids, nanobodies, and fragments, derivatives, and modifications thereof. Preferably the promoter is an inducible promoter and expressing comprises inducing the inducible promoter with an inducing agent.

Preferably isolating comprises chromatography, wherein the chromatography comprises a sulfate resin, a gel resin, an active sulfated resin, a phosphate resin, a heparin resin, or a heparin-like resin. Preferably the peptide isolated is conjugated with polyethylene glycol (PEG) and/or a derivative of PEG, or coupled to a polymer such as, for example, a polysaccharide, a peptide, an antibody or portion of an antibody, a lipid, a fatty acid, small molecule, hapten or a combination thereof.

Another embodiment of the invention is directed to an E. coli cell line containing a gor mutation. Preferably the cell line comprises cells obtained or derived from ATCC Deposit number PTA-126975.

Another embodiment of the invention is directed to methods of producing a protein comprising: expressing a preprotein in a recombinant cell which contains a recombinantly engineered protease gene. Preferably the protease gene and/or the preprotein gene contains a promotor and/or a translation induction sequence. The promoters may be the same of different and the translation induction sequences, if present, may be the same or different for the genes. Preferably after expression of the preprotein, expression of the protease gene is induced such that the preprotein is cleaved to form the protein; and harvesting the protein. Preferably the preprotein is selected from the group consisting of pro-insulin, pro-insulin-like proteins, prorelaxin, proopiomelanocortin, a proenzyme, a prohormones, proangiotensinogen, protrypsinogen, prochymotrypsinogen, propepsinogen, proproteins of the coagulation system, prothrombin, proplasminogen, proproteins of the compliment system, procaspases, propacifastin, proelastase, prolipase, procarboxypolypeptidases, proteins containing a cleavable leader sequence, cleavable tag sequences, proteins containing a cleavable extra N- or C-terminal amino acid (e.g., Met). Preferably the protease gene is integrated into the genome of the recombinant cell. Also preferably, a methionine aminopeptidase gene is integrated into the genome of the recombinant cell, wherein expression of the methionine aminopeptidase gene removes an N-terminal methionine from the preprotein or the protein. The expression of the methionine aminopeptidase gene is preferably under the control of an inducer sequence and the inducer sequence of the methionine aminopeptidase and the translation induction sequence of the preprotein may be the same or different. Also preferably, the recombinant cell has a reduced activity of one or more disulfide reductase enzymes which may be and E. coli with a gor mutation.

Another embodiment of the invention is directed to a recombinant cell line containing a methionine aminopeptidase gene and a protease gene, both of which are integrated. Preferably the cell further contains a reduced activity of one or more disulfide reductase enzymes, which may be attributed to a gor mutation.

Other embodiments and advantages of the invention are set forth in part in the description, which follows, and in part, may be obvious from this description, or may be learned from the practice of the invention.

### DESCRIPTION OF THE INVENTION

Proteins in E. coli are typically expressed with a methionine at the N-terminus because the correspondent ATG codon is required for initiation of translation. Proteins expressed intracellularly, therefore, contain N-terminal Methionine that is not part of the native amino acid sequence (unless the native sequence begins with a methionine). Removal of the N terminal methionine by MAP can be important for the function and stability of proteins. An endogenous methionine aminopeptidase (MAP) can cleave the N terminal methionine of newly synthesized protein, typically up to 60-70%. However, in highly expressed recombinant proteins, the level of activities of the endogenous MAP may not be sufficient to remove a desired amount of the N-terminal methionine. Removing N-terminal Methionine would be a significant issue in producing intracellular recombinant proteins in E. coli. Thus, E. coli strains that can efficiently cleave unwanted N-terminal Met would be highly desirable.

Solubility and proper folding of recombinant proteins expressed intracellularly is of a concern for E. coli expression systems. E. coli cytoplasm has a reducing environment that does not favor disulfide bond forming. As a result, recombinant proteins containing disulfide bonds are usually insoluble when expressed intracellularly. Purification of these insoluble proteins can be difficult, expensive and time consuming. High cell densities are preferable for the production of recombinant proteins, especially for commercial use.

Microorganisms genetically engineered to express large quantities of properly configured recombinant protein that also effectively remove N-terminal formyl-methionine have been surprisingly developed. These microorganisms are genetically engineered to express soluble recombinant proteins containing disulfide bonds in the cytoplasm and to remove N-terminal Methionine. These microorganisms produce a large quantity of the properly folded intracellular recombinant protein containing disulfide bonds without Methionine at the protein’s N-terminus.

Preferably, microorganisms contain one or more methionine aminopeptidase (MAP) genes incorporated into the genome of the bacteria. Peptidases that remove an N-terminal methionine include, but are not limited to, E. coli MAPs, Yeast MAPs and human MAPs, and their mutants, all of which can be utilized. The coding sequence of the MAP, under the control of an inducible promoter, was inserted into the bacterial genome, preferably in a manner as to prevent disruption of the genome. Having an inducible promoter allows the initiation of the expression of additional MAP at a selected time, preferably only when more MAP is needed to effectively remove formyl-methionine from overexpressed recombinant protein. The promoter for the MAP gene can be the same or different from the promoter used for the recombinant protein. In one example, the tac-promoter was utilized as a lactose/IPTG inducible promoter for both the MAP gene and the recombinant protein so the expression of the MAP and the recombinant protein can be induced at the same time. Different combinations of inducible promoters for expression of MAP and for that of the recombinant protein can be used to regulate the timing of the expression of each.

Incorporation of additional MAP into the genome is particularly desirable as the stable bacterial expression strains created can be used for the intracellular production of recombinant proteins with unwanted f-Met cleaved in vivo. Previously, removal of unwanted N terminal methionine has been done by post translationally in vitro digestion using purified MAP or by co-expression of MAP in the same vector as the recombinant protein or using an additional vector. The process is long and complicated. By using a cell line that can cleave f-met on demand, in vivo, these microorganisms greatly simplify protein expression and purification process.

MAPs cleave the N-terminal Methionine with specific requirements for the adjacent amino acids. The use of at least one additional MAP may allow the more efficient production of intracellular proteins without N terminal Methionine. If more than one MAP is inserted, they can be the same or different MAP genes. The transcriptions of these MAP genes may be under the same or different inducible promoters. These promoters may be the same or different from the promoter used to express the recombinant proteins. A combination of inducible promoters can be used to control the timing of and the amount of the production of intracellularly expressed recombinant protein without N terminal methionine.

E. coli strains capable of expressing soluble, properly folded intracellular recombinant proteins containing disulfide bonds have been described (see U.S. Pat. Nos. 10,597,664 and 10,093,704). An example of such a strain is the E. coli (BL21 Gor-). BL21Gor- has been used to express soluble, properly folded intracellular recombinant proteins containing disulfide bonds at high levels, including the vaccine carrier protein CRM₁₉₇, a genetically detoxified diphtheria toxin. Approximately 60% of the CRM₁₉₇ expressed in these cells contained an N-terminal methionine. The insertion of an additional MAP gene under the control of a promoter into such a strain allows for the production of soluble, properly folded intracellular recombinant proteins containing disulfide bonds and without N-terminal Methionine. An example of such a strain is the E. coli (BL21 Gor/Met) strain. This strain can produce intracellularly soluble proteins, with disulfide bonds and without N-terminal Methionine, in grams quantity per liter of cell culture. CRM₁₉₇ expressed in BL21 Gor/Met cells contained very low levels of N-terminal Methionine. Furthermore, the incorporation of the inducible methionine aminopeptidase gene into the E. coli genome did not significantly affect CRM₁₉₇ expression levels.

The MAP gene was inserted into the genome by homologous recombination, although several other options to facilitate insertion are available. The approach used for the creation of the Gor/Met cell strain is an example of gene insertion. For Gor/Met cells, red recombinase system was used to insert the MAP gene into Gor locus in BL21 Gor- cells. The MAP gene was cut from the BL21 genome using PCR and put under the control of Tac promoter and downstream of a chloramphenicol acetyltransferase (CAT) gene flanked by two short flippase recognition target (FRT) sequences. MAP and CAT gene together formed a transfer gene cassette. Fifty bases each of sequences flanking upstream and downstream of the original Gor locus were added to the transfer cassette upstream and downstream, respectively, by PCR. The final PCR product was used to transform to BL21 Gor- cell line that had already transformed with Red recombinase. The expression of red recombinase in the cell facilitated the homologous recombination of the sequences flanking Gor locus with the transfer cassette. Bacterial colonies that were resistant to chloramphenicol were confirmed to have successful transfer cassette insertion. Confirmed bacteria were subsequently transformed with flippase gene whose expression recognized FRT sequence flanking the CAT gene and clipped the gene away to leave the MAP inserted. Thus, the Gor/Met cell line has a MAP gene located at the Gor locus, without disturbing other parts of the genome.

To produce large quantities of protein, such as CRM₁₉₇ from E. coli host cells, an f-Met that is present at the N-terminus of the protein is enzymatically removed in the cytoplasm. Production quantities are typically quantified as mg/L of bacterial cell culture. Protein production achieved 25 mg/L or more, 50 mg/L or more 100 mg/L or more, 200 mg/L or more, 300 mg/L or more, 400 mg/L or more, 500 mg/L or more, 600 mg/L or more, 700 mg/L or more, 800 mg/L or more, 900 mg/L or more, 1,000 mg/L or more, 1,500 mg/L or more, or 2,000 mg/L or more. Protein expressed, as desired, include both full length and/or truncated proteins, as well as modified amino acid sequences of the protein. Modifications include one or more of conservative amino acid deletions, substitution and/or additions. A conservative modification is one that maintains the functional activity and/or immunogenicity of the molecule, although the activity and/or immunogenicity may be increased or decreased. Examples of conservative modifications include, but are not limited to amino acid modifications (e.g., single, double and otherwise short amino acid additions, deletions and/or substitutions), modifications outside of the active or functional sequence, residues that are accessible for conjugation in forming a vaccine, modifications due to serotype variations, modifications that increase immunogenicity or increase conjugation efficiency, modification that do not substantially alter binding to heparin, modifications that maintain proper folding or three dimensional structure, and/or modifications that do not significantly alter immunogenicity of the protein or the portions of the protein that provide protective immunity.

Recombinant cells used are preferably E. coli bacteria and, preferably, E. coli that are genetically engineered to shift the redox state of the cytoplasm to a more oxidative state such as, for example, by mutation of one or more disulfide reductase genes such as, for example, an oxidoreductase, a dihydrofolate reductase, a thioredoxin reductase, a glutamate cysteine lyase, a disulfide reductase, a protein reductase, and/or a glutathione reductase. Preferably one or more disulfide reductase genes are mutated and rendered non-functional or marginally functional such that the redox state of the cytoplasm of the cell is shifted to a more oxidative state as compared to wild type without compromising viability. Oxidative protein folding involves the formation and isomerization of disulfide bridges and plays a key role in the stability and solubility of many proteins including CRM₁₉₇. Formation and the breakage of disulfide bridges is generally catalyzed by thiol-disulfide oxidoreductases. These enzymes are characterized by one or more Trx folds that consist of a four-stranded β-sheet surrounded by three α-helices, with a CXXC redox active-site motif. The assembly of various Trx modules has been used to build the different thiol oxidoreductases found in prokaryotic and in eukaryotic organisms. In the bacterial periplasm, the proteins are kept in the appropriate oxidation state by a combined action of the couples DsbB-DsbA and DsbD- DsbC/DsbE/DsbG. Protein expression systems are well known in the art and commercially available. Also preferred are E. coli expression strainsthat expresses constitutively a chromosomal copy of the disulfide bond isomerase DsbC. DsbC promotes the correction of mis-oxidized proteins into their correct form. Cytoplasmic DsbC is also a chaperone that can assist in the folding of proteins that do not require disulfide bonds.

Recombinant bacteria containing expressible protein sequences, wherein an f-Met that is present at the N-terminus of the newly expressed protein is enzymatically removed. Preferred host cells include, but are not limited to, cells genetically engineered to shift the redox state of the cytoplasm to a more oxidative state, that contain and express an inducible MAP gene. Preferred cells are prokaryotes such as E. coli expression systems, Bacillus subtillis expression and other bacterial cellular expression systems. Preferably the cells contain a protein expression system for expressing foreign or non-native sequences. Also preferable, the sequences to be expressed are comprised of an expression vector which contains one or more of an inducible promoter (e.g., inducible preferably with specific media), a start codon (e.g., ATG), a ribosome binding site, and/or a modified sequence between ribosome binding site and ATG starting codon, or between start codon and the sequence to be expressed. Preferred modified sequences or spacer sequences include, for example, a number of nucleotides more or less than 9 (e.g., between 7 and 12 nucleotides), and preferably not 9 nucleotides.

It has also been surprisingly discovered that recombinant cells can be developed containing additional proteases that effectively cleave one or more different pre-proteins and/or pre-proproteins from the inactive to the active configuration. These proteins are generally referred to as zymogens (e.g., proenzymes) requiring post-translational modifications. Protein precursors are often used by a cell when the active protein is harmful, but needs to be expressed. By integrating these proteins into a recombinant cell, expression can be achieved safely and cost-effectively, and in large quantities. A protease gene which expresses a protease that performs the specific cleavage from inactive to active can be integrated into the cellular genome or transformed with a vector containing the protease gene of interest, all as described herein. The introduced protease gene can be placed under the control of a promotor in common with the recombinant gene to be expressed and collected, and the protease gene which clips of the methionine, or the protease gene may have a different promotor. Similarly, the gene may be inducible, either separately or induced basically simultaneously the recombinant gene to be expressed and collected, and the protease gene which clips of the methionine. Preferably, when expression of the recombinant protein is sufficiently done, the second protease would be activated and process the pro-protein to an active state. Preproteins where this would be effective in both save both time and cost include, but are not limited to pro-insulin to insulin, pro-insulin-like proteins to insulin-like proteins, prorelaxin to relaxin, proopiomelanocortin to opiomelanocortin, pro-enzymes to enzymes, and prohormones to hormones, and also removal of signal peptides, leader sequences, tags, etc., from a protein. In general, the protease introduced will be specific to the protein to be cleaved. Additional proteins which could be efficiently produced in this was include, but are not limited to angiotensinogen, trypsinogen, chymotrypsinogen, pepsinogen, proteins of the coagulation system (e.g., prothrombin, plasminogen), proteins of the compliment system, procaspases, pacifastin, proelastase, prolipase, procarboxypolypeptidases. In addition, certain genes can be modified to include a portion (e.g., leader or tag or internal sequence), that allows the protein to be expressed in an inactive form, which is only transformed into an active form upon being cleaved with a protease whose gene has also be introduced to the cell and subsequently activated.

By way of example, the gene of interest is inserted into the genome with a different promoter. The cell is induced to express that gene, which has disulfide bonds, and also the methionine peptidase, which trims off the methionine. Once a suitable amount of trimmed protein is produced in the cytoplasm, the second promoter is induced which processes the protein to its final or active form. Expression of active protein such as trypsin during growth would chew up a lot of needed proteins in the cytoplasm and interfere with expression of the recombinant protein. This approach would avoid the need for in vitro processing of the expressed pro-protein.

Another embodiment of the invention is directed to recombinant protein that is expressed in E. coli or another host cell using an expression vector with an inducible promoter and/or a modified sequence between ribosome binding site and ATG starting codon, cells wherein an f-met that is present at the N-terminus of the recombinant protein that is enzymatically removed. Preferably, the expression vector includes the lactose/IPTG inducible promoter, preferably a tac promoter, and the sequence between ribosome binding site and ATG starting codon.

Another embodiment of the invention comprises an expression construction of nucleotide or amino acids sequences and with or without a regulatory region. Regulatory regions regulate protein expression by adding one or more sequences that promote nucleic acid recognition for increased expression (e.g., start codon, enzyme binding site, translation or transcription factor binding site) or for inhibited expression (e.g., operators). Preferably, a regulatory element of the invention contains a ribosome binding site with a start codon upstream of and with a coding sequence that differs from the coding sequence of the recombinant protein.

Another embodiment of the invention is directed to proteins and peptides as well as portions and domains thereof, that can be manufactured according to the methods disclosed herein. Proteins and peptides comprise, but are not limited to, for example, those proteins and peptides that can be cytoplasmically expressed without leader or tag sequences and at commercially significant levels according to the methods disclosed and described herein. Preferably, these proteins and peptides show proper folding upon expression in recombinant cells of the invention. Recombinant cells of the invention preferably show reduced activity of one or more disulfide reductase enzymes, preferable reduced activity of less than five disulfide reductase enzymes, preferable reduced activity of less than four disulfide reductase enzymes, and preferable reduced activity of less than three disulfide reductase enzymes. Preferably expression of the proteins and peptides is increased in recombinant cells of the invention but may be not reduced or not significantly reduced compared with expression in recombinant cell that does not have reduced activity of one or more disulfide reductase enzymes. Proteins and peptides that can be expressed in the methods disclosed herein include, but are not limited to, for example, tetanus toxin, tetanus toxin heavy chain proteins, diphtheria toxoid, CRM, tetanus toxoid, Pseudomonas exoprotein A, Pseudomonas aeruginosa toxoid, Bordetella pertussis toxoid, Clostridium perfringens toxoid, Escherichia coli heat-labile toxin B subunit, Neisseria meningitidis outer membrane complex, Hemophilus influenzae protein D, Flagellin Fli C, Horseshoe crab Haemocyanin, and fragments, derivatives, and modifications thereof.

Another embodiment of the invention is directed to portions and domains of proteins that are expressed thereof, fused genetically or by chemical modification or conjugation (e.g., carbodiimide, 1-cyanodimethylaminopyridinium tetrafluoroborate (CDAP)) with another molecule. Preferred other molecules are molecules such as, but not limited to, other proteins, peptides, lipids, fatty acids, saccharides and/or polysaccharides, including molecules that extend half-life (e.g., PEG, antibody fragments such as Fc fragments), stimulate and/or increase immunogenicity, or reduce or eliminate immunogenicity.

Many proteins contain an N-terminal serine or threonine or may be genetically expressed with an N-terminal serine or threonine. An N-terminal serine or threonine can be selectively activated making it useful for conjugation. The presence of an N-terminal Methionine would block the ability of these amino acids to be selectively activated. The method described in this patent allow for the N-terminal Methionine to be cleaved allowing for the protein to be produced with the desired N-terminal amino acid. Typical conjugation partner molecules include, but are not limited to polymers such as, for example, bacterial polysaccharides, polysaccharides derived from yeast, parasite and/or other microorganisms, polyethylene glycol (PEG) and PEG derivatives and modifications, dextrans, and derivatives, modified, fragments and derivatives of dextrans. One example of a conjugated compound is PEGASYS® (peginterferon alfa-2a). Other polymers, such as dextran, also increase the half-life of proteins and reduce immunogenicity of the conjugate partner. Polymers may be linked randomly or directed through site specific conjugation such as, for example, by modification of N-terminal serine and/or threonine. Also, modifications may be used that selectively oxidize chemical groups for site specific conjugation.

Another embodiment of the invention is directed to methods of producing a peptide containing a domain, fragment and/or portion comprising: expressing the peptide from a recombinant cell containing an expression vector that encodes the peptide, wherein the recombinant cell has a reduced activity of one or more disulfide reductase enzymes and the expression vector contains a promoter functionally linked to a coding region of the peptide, wherein the one or more disulfide reductase enzymes comprises one or more of an oxidoreductase, a dihydrofolate reductase, a thioredoxin reductase, or a glutathione reductase; and isolating the peptide expressed, wherein the peptide expressed is soluble and wherein the protein or peptide is expressed with an f-met at the N-terminus that is removed by a peptidase that is also expressed within the recombinant cell. Preferably the expression vector contains a ribosome binding site, an initiation codon, and, optionally, an expression enhancer/repressor region. Preferably the recombinant cell has a reduced activity of only one disulfide reductase enzyme, only two disulfide reductase enzymes, or two or more disulfide reductase enzymes. Preferably the reduced activity of the disulfide reductase enzymes results in a shift the redox status of the cytoplasm to a more oxidative state as compared to a recombinant cell that does not have reduced activity of one or more disulfide reductase enzymes. Preferably the recombinant cell is an E. coli cell or a derivative or strain of E. coli. Preferably the soluble peptide expressed comprises a natively folded protein or domain of the protein. The promoter may be a constitutive or inducible promoter, whereby expression comprises inducing the inducible promoter with an inducing agent. Preferred inducing agents include, for example, lactose (PLac), isopropyl β-D-1-thiogalactopyranoside (IPTG), substrates and derivative of substrates. In one preferred embodiment, the genome of the recombinant cell contains an additional gene that preferably contains a coding region for a peptidase that preferably acts upon and selectively cleaves the peptide or protein expressed from the expression vector. Preferably the recombinant protein expression vector contains the same or a different inducible promoter as the MAP gene that has been inserted into the genome. The additional gene and the gene in expression vectors may be induced together with the same inducing agent, or with different inducing agents, optionally at different times depending on the promoters. Preferably the peptidase acts on and cleaves the peptide co-expressed with the peptidase. Preferably the peptide expressed is conjugated with a polymer such as, for example, dextran, a bacterial capsular polysaccharide, polyethylene glycol (PEG), or a fragment, derivative or modification thereof. Preferably the peptide expressed is coupled with a polymer which includes, for example, a polysaccharide, a peptide, an antibody or portion of an antibody, a lipid, a fatty acid, or a combination thereof.

Another embodiment of the invention comprises conjugates of proteins expressed and cleaved according to the disclosures herein including fragments, domains, and portions thereof as disclosed and described herein.

Another embodiment of the invention comprises fusion molecules of proteins included fragments, domains, and portions thereof as disclosed and described herein.

Another embodiment of the invention comprises a vaccine of proteins included fragments, domains, and portions thereof, as disclosed and described herein.

The following examples illustrate embodiments of the invention but should not be viewed as limiting the scope of the invention.

### Example 1 Insertion of MAP Gene Into Various Loci of the Bacterial Genome

Efficiently removal of the N-terminal Methionine in overexpressed intracellular recombinant proteins was achieved by inserting a MAP gene with a promoter, preferably inducible, into the E. coli genome. In this way a permanent cell line was created that expresses the MAP gene under the control of an inducer. A recombinant gene is cloned into the cell, also under control of an inducer. Thus, the MAP gene can be expressed at the desired time to efficiently cleave the N-terminal Methionine from the expressed recombinant protein. Many MAP enzymes other than E. coli MAP are known or have been devised. MAP from other species may have different selectivity for the adjacent amino acid. Some MAPs have been genetically altered to be less stringent in their requirement for a non-bulky amino acid adjacent to the N-terminal Methionine. The insertion of one or more of these MAPs with an inducible promoter into the E. coli genome would expand the range of N-terminal sequences which could be efficiently processed.

Inserting a recombinant gene into a genome may disrupt the E. coli genome structure, possibly impairing cell growth. A safe way to insert the MAP gene into the E, coli genome is to use a viable strain from which a gene has been deleted and to substitute in the MAP gene for the deleted gene. In this way, by replacing a gene for a gene, the probability of disruption of the genome can be reduced. Two strains of E. coli are widely used to manipulate genes and express recombinant proteins: the K12 strain and the B strain. E. coli strains, including the K12 and B strains, can be used as the host cell for the insertion of the MAP gene.

Insertion at the wrong site might be lethal to the bacteria, such as an insertion deletion in an open reading frame of an essential gene or at a site which disrupts control elements. Three illustrative protocols to insert the MAP gene, with an inducible promoter and a terminator are:

Insertion of the MAP gene upstream or downstream of an already defined gene. For example, the recombinant MAP gene can be inserted downstream of the endogenous MAP gene. The E. coli MAP gene has been studied and the gene structure has been defined. Insertion of the recombinant gene downstream will not disturb the expression of other gene.

Insertion of the MAP gene into a gene locus that has been previously deleted. The creation of the Gor/Met cell line is an example of the insertion of the MAP gene into the site of a deleted gene. Gor- cells were created by deleting the Gor gene in BL21 cells. The Gor/Met cell line was created by insertion of the MAP gene into the Gor locus of BL21 Gor- cells.

Insertion/replacement of a nonessential gene. As an example of the third method, the MAP gene is inserted to replace the T7 RNA polymerase in BL21(DE3) cells. BL21 (DE3) encodes a very active T7 RNA polymerase in the DE3 fragment which can transcribe recombinant genes under the control of T7 promoter. T7 polymerase gene can be replaced by MAP gene if the recombinant gene is transcribed by intrinsic RNA Polymerase under the control of T5 promoters.

Site directed mutagenesis techniques are required for the above-mentioned insertion site selection. Many methods are known to manipulate the bacterial genome and two examples are disclosed here. The most common method for the insertion/deletion of a gene is Red Recombineering. This technique has been used widely for mutations in bacteria genomes as well as eukaryotic genomes and starts with using PCR to introduce short sequences of DNA complementary to upstream and downstream of the selected site of insertion flanking the gene of interest. The PCR product is then electroporated into E. coli that has already expressed red recombinase in a previously transformed temperature sensitive vector. The red recombinase aids in the homologous recombination which inserts the gene at the selected site. Red recombinase can be removed by growing the bacteria at 42° C. since Red recombinase gene is on a temperature sensitive plasmid.

To enhance the selection for positive clones, a marker gene can be introduced and later removed after positive clones are confirmed. In one method, a flippase recognition signal is introduced to flank a marker gene, such as an antibiotic gene, that cloned downstream of inserted gene. The PCR product that was used for gene insertion will then include the marker gene. After the recombination event occurred, the marker gene can be used for positive clone selection. Once the positive clone is confirmed, flippase expression is introduced to the bacteria to remove the marker gene between two flippase recognition sites. This kind of insertion is marked with a scar that contains flippase recognition sequences at the insertion site. This method was used to create the Gor/Met E. coli strain by inserting the Met gene into the deleted Gor gene and is described in Example 2 below.

CRISPR technology can be used in E. coli when combined with Red recombineering. In the CRISPR-assisted red recombineering, two plasmids and one oligos are utilized. One plasmid encodes constitutively expressed Red recombinase and Cas9 protease. Another encodes the CRISPR guide RNA with the insertion site cloned in. These two plasmids have two compatible replication-of-origins. The oligo is made to contain the gene of interest flanked by the insertion site sequences. E. coli cells are transformed with these three elements and the surviving colonies should be the one that have the gene inserted: Cas9 protease will bind with CRISPR guide RNA to scan for the insertion site. Once the insertion site is located, Cas9 will cleave its double stranded DNA and allow the red recombinase to come in to perform homologous recombination between the cleaved site and the oligo with protein of interest. Those colonies with original sequence will be recognized and eliminated. Only the ones have the gene of interest will survive. The CRISPR assisted Red recombineering has a 65% success rate, while the other 35% comes from the failure of Cas9 to locate the insertion site. Thus, screening for positive clones is fast and straightforward.

### Example 2. Construction of an E Coli Cell Strain With a Gene Replacement for the Cytoplasmic Expression of Recombinant Proteins Without N-terminal Methionine

The construction of E. coli cell strain BL21 Gor- is described in U.S. Pat. Nos. 10,093,704 and 10,597,664. The strains described have the Gor gene deleted and an oxidative cytoplasm, such that proteins are expressed cytoplasmically, with properly folded disulfide bonds, and at high levels. To these stains, an extra E. coli MAP gene was inserted into the Gor gene locus and the new strain called Gor/Met E. coli. A Tac promoter (with a Lac operator) was added upstream of the MAP gene, so that expression of the MAP gene can be regulated by the timing of IPTG addition. This was accomplished as follows: An additional E. coli MAP gene was inserted into genome by homologous recombination. In the case of Gor/Met cells, the red recombinase system was used to aid in the insertion of the MAP gene into the Gor locus in BL21 Gor- cells. The MAP gene was PCR amplified from the BL21 Gor- genome and put under the control of a Tac promoter. The gene was then cloned downstream of a chloramphenicol acetyltransferase (CAT) gene flanked by two short flippase recognition target (FRT) sequences. The MAP and CAT gene together formed a transfer gene cassette. PCR was used to introduce fifty bases each of sequences flanking upstream and downstream of the original Gor locus into the transfer cassette upstream and downstream, respectively. The final PCR product was used to transform to BL21 Gor- cells previously transformed with Red recombinase. The expression of Red recombinase in the cell accelerated the homologous recombination of the sequences flanking Gor locus with that of transfer cassette. Bacterial colonies that were resistant to chloramphenicol were confirmed to have transfer cassette insertion. Confirmed bacteria were subsequently transformed with flippase gene whose expression recognized FRT sequence flanking the CAT gene and clipped the gene away to leave the MAP still inserted. Thus, the Gor/Met cell line has MAP gene inserted at the Gor locus, without disturbing other parts of the genome. This cell line was deposited strain and deposited with the American Type Culture Collection as Deposit No. PTA-126975 on Feb. 09, 2021. The results of sequencing using primers designed to flank the Gor locus confirmed the successful insertion of the MAP gene.

To demonstrate the functionality of the strain, several genes were expressed in BL21 Gor/Met cells and confirmed to efficiently cleave the N-terminal Methionine to produce the native protein.

### Example 3. Expression of CRM₁₉₇ in Gor/Met E. coli

CRM₁₉₇ is an enzymatically inactive and nontoxic form of diphtheria toxin that contains a single amino acid substitution G52E. Like DT, CRM₁₉₇ has two disulfide bonds. One disulfide joins Cys186 to Cys201, linking fragment A to fragment B. A second disulfide bridge joins Cys461 to Cys471 within fragment B. CRM₁₉₇ is commonly used as the carrier protein for carbohydrate-, peptide- and hapten-protein conjugates. As a carrier protein, CRM₁₉₇ has a number of advantages over diphtheria toxoid as well as other toxoided proteins.

Although CRM₁₉₇ has been produced in the original host Corynebacterium, a slow growing bacteria with a doubling time of hours instead of minutes, yields are low, typically <50 mg/L. Corynebacterium strains have been engineered to produce CRM₁₉₇ at higher levels (e.g., see U.S. Pat. No. 5,614,382). CRM₁₉₇ has also been expressed in a strain of Pseudomonas fluorescens at a high level. However, production of CRM₁₉₇ in a strain that is at a BL1 safety level and is inexpensive to culture and propagate would be advantageous. Expression of soluble, properly folded intracellular CRM₁₉₇ in BL21 Gor- strain has been successful, with >2 g CRM₁₉₇ per liter fermenter cell culture. However, the majority of the CRM₁₉₇ produced was found to have N-terminal f-methionine. The CRM₁₉₇ gene with a tac promoter was cloned into the Gor/Met E coli strain (e.g., see U.S. Pat. Nos. 10,093,704 and 10,597,664 for the gor- strain). Thus, both the MAP gene and the recombinant CRM₁₉₇ gene were under the control of the same tac promoters, capable of being expressed simultaneously upon IPTG induction.

Expression of CRM₁₉₇ in BL21 gor- and BL21 Gor/Met E. coli were compared. Similar yields, ~ 2 g/L were found for both strains, indicating that co-expression of the MAP gene did not significantly affect the expression of the CRM₁₉₇. Purified CRM₁₉₇ from BL21 gor- and BL21 Gor/Met strains were analyzed by MALDI-TOF mass spectrometry and the results summarized in Table 1. Lot NO21p114 was expressed in the Gor- strain and Lot NO21p221 was expressed in the Gor/Met strain. CRM₁₉₇ expressed in BL21 Gor- contained N terminal Met whereas CRM₁₉₇ expressed in Gor/Met E. coli did not, demonstrating that the method described in this invention was successful.

TABLE 1

Non-reduced CRM197 (n=3,1SD)

| Species | Theoretical | Observed | |
|---|---|---|---|
| | | NO21p114 (gor-) | NO21p221(Gor/Met) |
| CRM (w/out Met) | ~58,409 Da | 58,411.6.00±0.4 Da | 58,411.4 ±0.0 Da |
| CRM (with Met) | ~58,540 Da | 58,542.2.00±0.1 Da | not observed |

### Example 4 Expression of Cytokine IL10 From Epstein-Barr Virus in the Gor/Met Strain

The IL10 gene, derived from the Epstein-Barr virus, was cloned and expressed as a soluble intracellular protein in Gor/Met E. coli. A metal affinity tag was included on the C-terminal to facilitate purification. The IL10, purified by IMAC and ion exchange chromatography was subjected to mass spectrometry analysis to determine the sequence of the N-terminal peptide. Following enzymatic digestion with trypsin, the sample was analyzed by LC-MS/MS, which found that the protein did not have an N-terminal methionine.

The procedure was carried out using the following protocol: The sample was digested with trypsin and analyzed by LC-MS/MS on a LTQ Orbitrap Velos (ThermoFisher Scientific, Bremen, Germany), interfaced with a Proxeon 1200 nanoLC (Proxeon Biosystems). The chromatography was performed on a 75 µm i.d. Self-Pack PicoFrit fused silica capillary column 15 cm in length (New Objective, Woburn, MA). The stationary phase was a reverse-phase C18 Jupiter column (5 µm, 300 Å) (Phenomenex, Torrance, CA). Mass resolution was set to 30 000 for parent mass determination in MS mode and to 7 500 for acquisition of the fragmentation spectra in MS/MS mode. MS/MS spectra obtained during the LC-MS/MS run were submitted to a Mascot search against the expected protein sequence. Carbamidomethyl (C) was selected as Fixed modifications, and Oxidation (M) was selected as Variable modifications. The objective was to retrieve from the digest solution the peptide corresponding to the first tryptic cleavage. This peptide, on the submitted sequence, would include the Arginine on position 13. If Methionine was present on the N-terminus, the result would be amino acid sequences 1 to 13 (MTDQCDNFPQMLR; SEQ ID NO: 1), while if Methionine would be absent, the results would be amino acid sequences 2 to 13 (TDQCDNFPQMLR; SEQ ID NO: 2).

TABLE 2

| | | | | | |
|---|---|---|---|---|---|
| 1 | MTDQCDNFPQ | MLRDLRDAFS | RVKTFFQTKD | ELDNLLLKES | LLEDFKGY LG |
| 51 | CQALSEMIQF | YLEEVMPQAE | NQDPEIKDHV | NSLGENLK TL | RLRLRRCHRF |
| 101 | LPCENKSKAV | EQIKNAFNKL | QEKGIYKAMS | EFDIFINYIE | AYMTIKAR GH |
| 151 | HHHHH (SEQ ID NO: 3) | | | | |

The amino acids underlined and bolded in Table 2 identify the sequences identified by Mascot from the MS/MS sequencing data. This signifies that ions were found to confirm the 2-13 sequence (no Methionine on the N-terminus) and that no ions were found for the 1-13 sequence (Methionine on the N— terminus).

The 2-13 sequence (without a Methionine on the N-terminus) was confirmed by the presence of the following ions in the MS trace: 762.8 m/z (+2), 508.9 m/z (+3), 770.8 m/z (+2), and their corresponding fragmentation pattern from the MS/MS trace. The three identified ions all contain an alkylated cysteine but the ion at 770.8 m/z also contains an oxidized methionine (position 11 on the submitted sequence). In conclusion, the IL10 expressed in Gor/Met was produced without the N-terminal Methionine.

### Example 4. Expression of a Genetically Detoxified Tetanus Toxin (8MTT) in Gor/Met E. Coli

Tetanus toxin is known as one of the most potent toxins for humans and referred to as a spasmogenic toxin, or TeNT. The LD50 of this toxin is measured to be approximately 2.5-3 ng/kg. Tetanus toxin is produced by Clostridium tetani, an anaerobic bacillus normally found in soil, as a single polypeptide chain that is post translationally cleaved by a trypsin-like protease into two chains to form the active protein. The light chain (LC), a 50 kDa domain, contains a N-terminal endopeptidase, the heavy chain (HC) contains a 50 kDa receptor binding domain on C terminus (HCC) and a 50 kDa LC translocation domain is located on the N terminus (HCN). The two chains are connected by a single disulfide bond. Tetanus toxin enters peripheral motor neurons by binding to gangliosides and synaptic proteins on their surface through the C-terminal domain of the heavy chain (HCC). The toxin traffics to the soma and to synapses of interneurons in the central nervous system, and transcytoses and enters inhibitory neurons in synaptic vesicles. In the inhibitory neurons, the heavy chain’s translocation domain (HCN) undergoes a pH-mediated conformational change and transports the LC through the membrane of synaptic vesicles into the cytoplasm where LC is released into the cell cytosol and cleaves vesicle-associated membrane protein 2 (VAMP2), a vesicle soluble NSF attachment protein receptor (SNARE). VAMP2 cleavage in inhibitory neurons blocks neurotransmitter exocytosis, preventing release of inhibitors of neuromuscular synapse function, leading to continued neuromuscular activation and spastic paralysis.

Chemically inactivated tetanus toxoid (TTxd), formed by treating the toxin with formaldehyde, is used as an effective vaccine against tetanus. TTxd is also used as a conjugate vaccine carrier for polysaccharide antigens. Conjugated vaccines using TTxd as the carrier protein include vaccines against Haemophilus influenzae type b and Neisseria meningiditis. However, TTxd has many of its amines, used for conjugation, blocked by the toxoiding process. Furthermore, TTxd is a heterogeneous product and contains aggregates, along with Clostridium and media contaminants. TTxd vaccine needs to be further purified for use in conjugate vaccines. More important, the production and purification of TT from the Clostridium is time consuming and costly. A genetically inactivated homogeneous recombinant Tetanus toxin, produced in a low-cost host like E. coli, would be desirable.

Different strategies of producing inactivated recombinant TT proteins as vaccines or carrier proteins have been explored. One is the use of heavy chain fragments (TTHC). Another is the use of genetically inactivated tetanus toxin (U.S. Pat. Application Publication No. 2020/03841201). TTHC is part of the TT that does not carry the catalytic domain. Neutralizing antibodies against the TTHC subunit vaccine was claimed to outperform full toxoid vaccine antibodies. TTHC was expressed at high levels (>400 mg/L) in the BL21 Gor- system (e.g., see U.S. Pat. Nos. 10,597,664 and 10,093,704).

8MTT is a genetically detoxified tetanus toxin (TT) with 8 amino acid mutations. Like tetanus toxin, 8MTT has 5 disulfide bonds. The LD50 is more than 50 million-fold less toxic than native TT. 8MTT vaccination elicited a strong immune response IgG antibody response in mice, is a lead candidate for a new tetanus vaccine and, has great potential to be used as a conjugate vaccine carrier protein, similar to the widely used CRM₁₉₇. 8MTT was originally cloned into the pET28 expression vector and expressed in BL21(DE3) cell with a His tag attached to facilitate the purification. The expression was about 10 mg/liter in the shaker flasks. To produce protein without the tag and without the N-terminal Methionine, 8MTT gene was subcloned into an expression vector with the tac promoter (with lac operator) and T7 terminator, and then expressed in BL21 Gor/Met cells in a fed-batch fermenter. The expressed M8TT protein was found to be soluble and could be purified at more than 500 mg per liter. Using a combination of anion exchange column, HIC column and TFF diafiltration/concentration M8TT was purified to more than 99% purity. The purified M8TT was analyzed by MALDI-ISD (Matrix Assisted Laser Desorption/Ionization - In Source Decay) to obtain terminal fragmentation. ISD allowed for the identification of a ladder of N-terminal fragments and confirmed that the sequence did not have an N-terminal Methionine and the first residue of the sequence was the expected Proline. Thus, the Gor/Met E. coli strain efficiently expressed large quantities of soluble M8TT without an N-terminal methionine.

### Example 5. CRM₁₉₇ Mutant with N-Terminal Serine

The CRM₁₉₇ gene containing an N-terminal serine (CRM-Ser) was cloned into the Gor/Met E. coli strain and grown and expressed in a bioreactor. Without any optimization of fermentation conditions >1 g/L of soluble CRM-Ser was expressed, showing that expression of the protein was excellent. The cells were harvested and CRM-Ser purified. The CRM-Ser was analyzed by MALDI-ISD as described in Example 4. ISD allowed for the identification of a ladder of N-terminal fragments and confirmed that the sequence did not have an N-terminal Methionine and the first residue of the sequence is the expected Serine. Thus, the Gor/Met E. coli strain efficiently expressed large quantities of soluble CRM-Ser without an N-terminal methionine. The Serine can be selectively oxidized and used for conjugation.

### Example 6: Possible MAP Genes to Insert in Bacterial Genome

Removal of N-terminal Methionine by MAP can be important for proper function and stability of proteins. Most of the recombinant proteins expressed in E. coli still have methionine starting codon on N terminus even though the intrinsic MAP is active. Insufficient MAP or its cofactors may be present when overexpressed recombinant proteins are produced. To ensure the processing of the N terminal methionine in recombinant proteins, extra MAP genes are inserted under strong, inducible promoters to facility methionine cleaving process when necessary.

### 1. E. coli MAP Gene

Gor/Met cell is an example to insert an extra E. coli MAP gene in bacteria genome and under the same inducible promoter as that of the recombinant gene on the expression vector. When not induced, this MAP gene remains silent and the bacteria propagate with no burden of extra gene expression. As only the recombinant protein is induced to be expressed that extra MAP protein are also induced, MAP protein can be designed to be turned on as needed. A potential drawback of this system is that not all proteins with N terminal methionine can be efficiently cleaved by E. coli MAP. E. coli MAP works when a small amino acid (G, A, S, C, P, T, V) (P1′ position) is adjacent to N-terminal Methionine. Preferably the P2′ position (the amino acid C terminal to P1) of amino acid is not proline. To process those proteins with bulky amino acids next to the N-terminal Methionine, other MAP genes with different P1 and P2 amino acid requirements maybe inserted instead.

### 2. Two Different MAP Gene in Tandem in Different Inducible Promoters

Yeast genes are processed by two MAP genes. Yeast MAP1 and MAP2 exhibit different cleavage efficiencies against the same substrates in vivo. Both MAPs were less efficient when the second residue was V, and MAP2 was less efficient than MAP1 when the second residue was G, C, or T. Humans also have two MAPs: MAP1 and MAP2. They can both process proteins containing A, C, G, P, or S at the P1′ position. When the P1′ residue is T or V, the N-terminal Met removal is primarily catalyzed by MAP2 and the extent of cleavage depends on the sequence at P2′-P5′ positions. When the P2′ residue is not A, G, or P, the N-terminal processing is expected to be complete. When A, G, or P is the P2′ residue, Methionine removal is either incomplete or does not occur. Since different MAP has different substrate specificity, two or more MAP genes from different or same species may be able to cover Methionine removing processing from more recombinant proteins. The (for example inducible) promoters that control the gene transcription maybe different so that two MAP genes can be turned on at different time, or one on/one off, depending on the need.

### 3. Insert a Mutated MAT Gene That Is Capable to Cleave All N Terminal Methionine Without Restriction on the Amino Acid Followed

The current MAPs disfavor some protein’s N terminal structures and will not catalyze the removal of their N terminal Methionine. A universal rule that predicts whether the initiating Methionine will be process by MAPs is based on the size of amino acid at the P1′ position. In general, if amino residues have a radius of gyration of 1.29 Å or less, Methionine is cleaved. For human MAPs, they have even more stringent requirement for substrates have acidic residues at the P2′ an P5′ position. To expand the substrate specificity of the existing MAPs, an E. coli MAP gene was mutated so that its product can cleave 85-90% of N terminal methionine on proteins listed in the protein database. This MAP has three mutations in its substrate binding pocket, thus allows removal of N-terminal Methionine from proteins with not only small amino acid but also bulky or acidic amino acid (e.g., M, H, D, N, E, Q, L, I, Y and W, at P2′ position). These enzymes can also cleave the amino acid at the P1′ position if amino acid residue at P2′ position is small. Insertion of this MAP gene processes proteins with broader N terminal structure. Again, adding an inducible promoter will be beneficial to control the activity of this powerful mutant gene expression.

### Example 7: Proteins Capable of Produced in the Gor/Met Cell Line Without Disulfide Bond

Although the Gor/Met cell strain was developed to express disulfide bond proteins without an N-terminal Methionine, proteins without disulfide bond can be expressed in Gor/Met cells without an N terminal methionine. These proteins can also be expressed in E. coli with a Met but not necessarily be gor-. An example of such a protein is Staphylococcus protein A (SPA) which is widely used in antibody purifications. SPA is a 42 kDa protein originally found in the cell wall of the bacteria Staphylococcus aureus. This protein is composed of five homologous Ig-binding domains that can bind proteins from many mammalian species, particularly IgGs and binds the heavy chain within the Fc regions of most immunoglobulins and within the Fab regions of the human VH3 family. SPA does not contain any disulfide bonds. Commercially, SPA comes from two sources: Staphylococcus aureus mutant strains that contains lesions in the cell wall where SPAs are secreted (Sigma), E. coli that expresses SPA as a recombinant protein, either intra- or extra-cellularly (Sigma-Aldrich, SinoBiological, ThermoFisher and DeNovo Biopharma, and others). SPA production can be at high levels in Pichia pastoris. The majority of SPA are expressed intracellularly E. coli. Since the mature SPA sequence starts with Alanine and not Methionine, for SPA to be produced intracellularly, either a methionine needs to be added to the gene at the N-terminus or it needs to be expressed at the C-terminus of a fusion protein which can be cleaved to release the mature protein in vitro. The former is not the true form of mature protein, the latter is not cost and time efficient.

The expression of SPA is efficient in bacterial strains disclosed herein that have an inducible MAP gene insertion. SPA is expressed in E. coli strains in high quantity, and functions similarly as in the Gor/Met cell line and can also be expressed in a MAP cell line. The advantage of using MAP cell line over other cell line is that the f-methionine can be cleaved at will. The inducible promoter of MAP gene can be expressed at the same time when SPA is expressed or anytime afterwards, by design. No in vitro manipulation is required to remove the N-terminal Methionine.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. A method of producing a protein containing one or more sulfide linkages comprising:
expressing the protein from a recombinant cell containing a genome and an expression vector that encodes the protein sequence, wherein the recombinant cell has a reduced activity of one or more disulfide reductase enzymes, and the N-terminus of the protein contains a methionine;
expressing a peptidase from a gene of the recombinant cell, wherein the peptidase removes the methionine from the N-terminus of the protein expressed; and
isolating the protein.

2. The method of claim 1, wherein the protein expressed comprises tetanus toxin, tetanus toxin heavy chain proteins, diphtheria toxoid, tetanus toxoid, Pseudomonas exoprotein A, Pseudomonas aeruginosa toxoid, Bordetella pertusis toxoid, Clostridium perfringens toxoid, Escherichia coli (E. coli) heat-labile toxin B subunit, Neisseria meningitidis outer membrane complex, Hemophilus influenzae protein D, Flagellin Fli C, Horseshoe crab Haemocyanin, or fragments, derivatives, or modifications thereof.

3. The method of claim 1, wherein the recombinant cell has a reduced activity of only one disulfide reductase enzyme.

4. The method of claim 1, wherein the reduced activity is of more than one disulfide reductase enzymes.

5. The method of claim 1, wherein the recombinant cell is an E. coli cell or a derivative or strain of E. coli.

6. The method of claim 5, wherein the recombinant cell is obtained or derived from ATCC Deposit No. PTA-126975.

7. The method of claim 1, wherein the peptidase comprises a methionine aminopeptidase.

8. The method of claim 1, wherein the expression vector contains a ribosome binding site, an initiation codon, and/or an expression enhancer region.

9. The method of claim 1, wherein the expression vector contains an inducible first promoter and expressing the protein comprises inducing the inducible first promoter with a first inducing agent.

10. The method of claim 1, wherein the gene contains an inducible second promoter and expressing the peptidase comprises inducing the inducible second promoter with a second inducing agent.

11. The method of claim 1, wherein the expression vector contains an inducible first promoter and expressing the protein comprises inducing the inducible first promoter with a first inducing agent, the gene contains an inducible second promoter and expressing the peptidase comprises inducing the inducible second promoter with a second inducing agent, and the first inducing agent and the second inducing agent are the same.

12. The method of claim 1, wherein the peptidase gene is integrated into the genome of the recombinant cell.

13. The method of claim 1, wherein isolating comprises chromatography.

14. The method of claim 13, wherein the chromatography comprises a sulfate resin, a gel resin, an active sulfated resin, a phosphate resin, a heparin resin or a heparin-like resin.

15. The method of claim 1, further comprising conjugating or coupling the isolated protein with a chemical compound.

16. The method of claim 15, wherein the chemical compound comprises a polysaccharide, a polymer, a polyethylene glycol, a derivative of polyethylene glycol, a peptide, an antibody or portion of an antibody, a lipid, a fatty acid, or a combination thereof.

17. A method of producing a peptide comprising:
expressing the peptide in a recombinant cell containing a gene that encodes a peptidase enzyme,
wherein the gene that encodes the peptidase enzyme is integrated into the genome of the recombinant cell,
wherein the recombinant cell has a reduced activity of one or more disulfide reductase enzymes, wherein the reduced activity of one or more disulfide reductase enzymes results in a shift the redox status of the cytoplasm to a more oxidative state as compared to a recombinant cell that does not have reduced activity of one or more disulfide reductase enzymes, and
wherein the peptide contains an N-terminal methionine;
expressing the peptidase enzyme which removes the N-terminal methionine from the peptide; and
isolating the peptide.

18. The method of claim 17, wherein the peptide comprises tetanus toxin, tetanus toxin heavy chain proteins, diphtheria toxoid, tetanus toxoid, Pseudomonas exoprotein A, Pseudomonas aeruginosa toxoid, Bordetella pertusis toxoid, Clostridium perfringens toxoid, Escherichia coli (E. coli) heat-labile toxin B subunit, Neisseria meningitidis outer membrane complex, Hemophilus influenzae protein D, Flagellin Fli C, Horseshoe crab Haemocyanin, or fragments, derivatives, or modifications thereof.

19. The method of claim 17, wherein the recombinant cell has a reduced activity of only one disulfide reductase enzyme.

20. The method of claim 17, wherein the recombinant cell has a reduced activity of two or more disulfide reductase enzymes.

21. The method of claim 17, wherein the one or more disulfide reductase enzymes comprises one or more of an oxidoreductase, a dihydrofolate reductase, a thioredoxin reductase, or a glutathione reductase.

22. The method of claim 17, wherein the recombinant cell is an E. coli cell or a derivative or strain of E. coli.

23. The method of claim 17, wherein a gene that encodes the peptide contains a first inducible promoter and/or a gene that encodes the peptidase enzyme contains a second inducible promoter.

24. The method of claim 17, wherein a gene that encodes the peptide contains a first inducible promoter and a gene that encodes the peptidase enzyme contains a second inducible promoters and the first and second inducible promoters are the same.

25. The method of claim 17, wherein isolating comprises chromatography.

26. The method of claim 25, wherein the chromatography comprises a sulfate resin, a gel resin, an active sulfated resin, a phosphate resin, a heparin resin or a heparin-like resin.

27. The method of claim 17, further comprising conjugating or coupling the isolated peptide with a chemical compound.

28. The method of claim 27, wherein the chemical compound comprises a polysaccharide, a polymer, a polyethylene glycol, a derivative of polyethylene glycol, a peptide, an antibody or portion of an antibody, a lipid, a fatty acid, or a combination thereof.

29. The method of claim 17, wherein the peptide is oxidized with an oxidizing agent.

30. The method of claim 29, wherein the oxidizing agent comprises a hydrazide, a hydrazine, an aminooxy group, N-terminal 1-amino, 2-alcohol amino acid, or a combination thereof.

31. A method of producing a peptide containing disulfide bonds comprising:
expressing the peptide in a recombinant cell containing a gene that encodes a peptidase enzyme,
wherein the peptide is encoded in an expression vector,
wherein the gene that encodes the peptidase enzyme is integrated into the genome of the recombinant cell,
wherein the recombinant cell has a reduced activity of one or more disulfide reductase enzymes,
wherein the recombinant cell is E. coli, and
wherein the peptide contains an N-terminal methionine;
expressing the peptidase enzyme which removes the N-terminal methionine from the peptide; and
isolating the peptide from within the cytoplasm of the recombinant cell, wherein the peptide isolated is soluble.

32. Recombinant cells obtained or derived from ATCC Deposit No. PTA-126975.

33. A method of producing a protein comprising:
expressing a preprotein in a recombinant cell which contains a recombinantly engineered protease gene containing a translation induction sequence;
inducing expression of the protease gene such that the preprotein is cleaved to form the protein; and
harvesting the protein.

34. The method of claim 33, wherein the preprotein is selected from the group consisting of pro-insulin, pro-insulin-like proteins, prorelaxin, proopiomelanocortin, a proenzyme, a prohormones, proangiotensinogen, protrypsinogen, prochymotrypsinogen, propepsinogen, proproteins of the coagulation system, prothrombin, proplasminogen, proproteins of the compliment system, procaspases, propacifastin, proelastase, prolipase, and procarboxypolypeptidases.

35. The method of claim 33, wherein the protease gene is integrated into the genome of the recombinant cell.

36. The method of claim 33, wherein a methionine aminopeptidase gene is integrated into the genome of the recombinant cell.

37. The method of claim 36, wherein expression of the methionine aminopeptidase gene removes an N-terminal methionine from the preprotein or the protein.

38. The method of claim 37, wherein expression of the methionine aminopeptidase gene is under the control of an inducer sequence.

39. The method of claim 38, wherein the inducer sequence of the methionine aminopeptidase and the translation induction sequence of the preprotein are different.

40. The method of claim 38, wherein the inducer sequence of the methionine aminopeptidase and the translation induction sequence of the preprotein are the same.

41. The method of claim 33, wherein the recombinant cell has a reduced activity of one or more disulfide reductase enzymes.

42. The method of claim 41, wherein the recombinant cell is E. coli that contains a gor mutation.

43. A recombinant cell line containing a methionine aminopeptidase gene and a protease gene, both of which are integrated.

44. The recombinant cell of claim 43, which has a reduced activity of one or more disulfide reductase enzymes.

45. The recombinant cell of claim 44, which contains a gor mutation.

46. A method of producing a peptide comprising:
expressing the peptide in a recombinant cell, wherein the expressed peptide contains an N-terminal methionine, and the recombinant cell contains a gene that encodes a peptidase;
expressing the peptidase gene such that the N-terminal methionine is cleaved from the expressed peptide; and
isolating the peptide.

47. The method of claim 46, wherein the peptide is expressed from another gene that is integrated into the genome of the recombinant cell.

48. The method of claim 46, wherein the peptidase gene is integrated into the genome of the recombinant cell.

49. The method of claim 46, wherein the peptidase is methionine amino peptidase.

50. The method of claim 46, wherein the recombinant cell is an E. coli cell.


SEQUENCE LISTING (3 sequences)

SEQ ID NO: 1 - protein, 13 aa, Artificial Sequence
  Description of Artificial Sequence: Synthetic peptide
MTDQCDNFPQMLR

SEQ ID NO: 2 - protein, 12 aa, Artificial Sequence
  Description of Artificial Sequence: Synthetic peptide
TDQCDNFPQMLR

SEQ ID NO: 3 - protein, 155 aa, Artificial Sequence
  Description of Artificial Sequence: Synthetic polypeptide
MTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGHHHHHH